(19) United States
(12) Patent Application Publication
(10) Pub. No.: US 2015/0313676 A1
(43) Pub. Date: Nov. 5, 2015

(54) WRISTED SURGICAL INSTRUMENT CAPABLE OF MULTIPLE FUNCTIONS, WITHOUT REQUIRING EXTRA INPUTS

(71) Applicant: Chinmay DEODHAR, (US)

(72) Inventor: Chinmay Deodhar, Pune (IN)

(21) Appl. No.: 14/422,993

(22) PCT Filed: Aug. 19, 2013
(86) PCT No.: PCT/IB2013/056728

§ 371 Date: Feb. 20, 2015

(30) Foreign Application Priority Data
Aug. 21, 2012 (IN) 2425/MUM/2012

(51) Int. Cl. A61B 19/00

(52) U.S. Cl. CPC A61B 19/2203; A61B 2019/2234; A61B 2019/2215

(57) ABSTRACT

The present invention is generally related to the art of surgical instruments for use in minimally invasive surgery and more specifically to robotic laparoscopic surgery. According to one aspect of the invention, this articulated instrument is capable of producing a wrist-like motion, i.e. the end effector generally has three degrees of freedom. Additionally, it also has another degree of freedom for actuation of the end effector. The end effector has a configurable set of arms which are capable of performing a first function in a first mode and a second function in a second mode. This end effector also has the capability to dynamically switch between two modes of different functions (e.g.: scissor and grasper). These actions of switching back and forth between the different modes (e.g.: scissor and grasper) can be described as extra actions. These extra actions are achieved without increasing the number of inputs.

### CROSS-REFERENCES TO RELATED APPLICATIONS

This application claims the benefit of Indian Provisional Application No. 2425/MUM/2012 entitled “COMBINATION OF SCISSOR, GRASPER AND ARTICULATED ROBOTIC SURGERY INSTRUMENT CAPABLE OF WRIST-LIKE MOTION”, filed 21 Aug. 2012, the entire disclosure of which is hereby incorporated herein by reference.

### FIELD OF THE INVENTION

The present invention is generally related to the art of surgical instruments for use in laparoscopic surgery and more specifically to an instrument capable of being used in robotic laparoscopic surgery.

### BACKGROUND OF THE INVENTION

Minimally invasive surgical (MIS) technologies possess a considerable number of advantages as compared to open surgical procedures. These include reduced blood loss during surgery, thereby lessening the need for blood transfusions. Smaller incisions made during such procedures may reduce pain, resulting in lesser need for pain medication. In comparison to patients treated with open surgical procedures, patients who have undergone minimally invasive surgeries may recover faster and have less post-operative scarring. Improvement in the existing minimally invasive surgical techniques may increase the use of such techniques and thus dramatically reduce surgical side-effects and hasten patient recovery periods.

Endoscopy may probably be the most common form of minimally invasive surgery. Commonly employed endoscopic techniques include arthroscopy, pelviscopy, hysteroscopy, laparoscopy and the like. Laparoscopy is possibly the most commonly used endoscopic technique. These surgeries are generally performed in abdominal or pelvic cavities and typically require a visual device called the laparoscope, as well as a variety of surgical instruments. In a routine laparoscopic surgery the abdominal cavity is insufflated with a gas such as carbon dioxide and a cannula is inserted through small incisions, to facilitate the entry of laparoscopic surgical instruments. Laparoscopic instruments are similar to those used for open surgeries, with the exception that these tools possess a long, thin, extension tube between the handle and the end effectors. As mentioned in this document, the end effectors of the tool refers to its functional part, which may include scissors, graspers, needle drivers, cauterizers, suction, irrigation, clip-appliers and the like. In routine laparoscopic surgeries, the instruments are passed through the trocar to the surgical site inside the abdominal cavity and maneuvered from outside the abdomen. The procedure is monitored by means of a screen displaying an enlarged image of the surgical site taken from the laparoscope.

MIS technologies are being augmented with tele-robotic surgical systems, to permit surgeons to operate on a patient from a distant location. Such improvements also increase the dexterity with which such tools can be used at a surgical working site. In a tele-robotic surgical system, the surgeon is provided with an ergonomically optimal position at a workstation, from where he or she can view a stereoscopic image of the surgical site on a screen. The hands of the surgeon fit into a “master” that serves as an interface between the surgeon and the computer, which servo-mechanically operates the motion of the surgical instrument. During the surgical procedure, the surgeon views the three dimensional image and operates on the patient by controlling the master input of the workstation. Surgical instruments with a variety of end effectors, such as tissue graspers, needle drivers, scissors, etc. can be controlled by the implementation of tele-robotic surgery, with the help of master control devices.

The development of the tele-robotic surgical system for minimally invasive surgeries enables tools to simulate the fluid wrist-like motions and overcome the motion limitations of non-robotic instruments. This is facilitated due to a wrist-like mechanism that allows the surgeon to use “master controls” to alter the position or orientation of the working end of the tool, relative to the end of the shaft. Such a tele-robotic surgical system generally has two robotic arms, each of which carries a surgical tool. There are typically two master controls, each of which is connected to a robotic arm with a surgical instrument. Each master control can be held and controlled by each of the two hands of the surgeon.

In many existing minimally invasive telesurgical robotic systems, manipulation of the surgical instruments is provided by a surgical robot having a number of robotic arms. Each of the robotic arms has a number of robotic joints and a mounting fixture for the attachment of a surgical instrument. Integrated in with at least one of the mounting fixtures are a number of drive couplers (e.g., rotary drive couplers) that drivingly interface with corresponding input couplers of the surgical instrument. The surgical instrument includes mechanisms that drivingly couple the input couplers with an associated motion of the surgical instrument (e.g., main shaft rotation, end effector pitch, end effector yaw, end effector jaw clamping). In many existing minimally invasive telesurgical robotic systems, there are four drive couplers integrated in with each of the mounting fixtures (e.g., one drive coupler to actuate main shaft rotation, one drive coupler to actuate end effector pitch, one drive coupler to actuate end effector yaw, and one drive coupler to actuate end effector jaw articulation).

A problem arises, however, when it is desired to employ a surgical robot having a number of output couplers per mounting fixture (e.g., four) to manipulate a surgical instrument having more than that number of functions (e.g., five such as main shaft rotation, end effector pitch, end effector yaw, end effector jaw grasping, and tissue shearing).

Thus, there is believed to be a need for surgical assemblies and related methods that employ a single input drive for two end effector functions (e.g., two different mechanisms). This problem has been only partially solved by WO2012166817, which discusses a surgical instrument architecture, where two different articulated members are actuated by a single link member. This may enable two different functions to be carried out, by two different articulating members, such as a grasper and a cutting blade.

However, it may not always be possible to have a different articulating member to carry out a different function. There are multi-functional instruments wherein the different functions are carried out by different configurations of the same set of articulated members, which can lock into different function modes, and need to be switched from one mode to another to perform one function from next.

WO2011161626 discloses one such device, where the same set of articulated members can lock into a scissor mode or a grasper mode, and there is a need for switching between these modes with no additional number of inputs. This instrument is designed to be used as a manual hand operated laparoscopic surgical device. However, one limitation of this invention is that it can only work as a straight conventional form of instrument, but not as an articulated, wristed instrument. This is due to the fact that the switching mechanism requires an action of a rigid hollow rod, which is not possible to bend over the articulated wristed joints. This patent describes a combination instrument capable of performing the functions of scissor and grasper, and has an articulated wrist.

### OBJECTS OF THE INVENTION

It is an object of this invention to provide for a multi-functional instrument that is also capable of wrist-like motion.

It is an object of this invention to provide for a multi-functional instrument, capable of a wrist-like motion, and which is capable of switching between different functions without requiring more number of inputs than are necessary to provide the individual functions and the wrist-like manipulation.

It is an object of this invention to combine scissor and grasper functionalities into a single end-effector instrument for minimally invasive surgery.

It is another object of this invention to combine scissor and grasper functionalities into a single end effector instrument for robotic minimally invasive surgical instrument.

Another object of this invention is to provide for the functions of scissor, grasper and the ability to switch between the scissor and grasper modes, without increasing the number of inputs than those required for wrist-like motion plus end-effector actuation. This is a critically important feature, because it enables compatibility with legacy robotic arm end couplings.

Another object of this invention is to provide for the above functions, without increasing the over-all diameter of the surgical instrument, as compared to conventional single function robotic surgical instruments.

### SUMMARY OF THE INVENTION

The present invention is generally related to the art of surgical instruments for use in minimally invasive surgery and more specifically to an instrument capable of being used in robotic laparoscopic surgery.

According to one aspect of the invention, this articulated instrument is capable of producing a wrist-like motion, i.e. the end effector generally has three degrees of freedom. Additionally, it also has another degree of freedom for actuation of the end effector, which has a configurable set of arms which are capable of performing a first function in a first mode and a second function in a second mode. This end effector also has the capability to dynamically switch between two modes of different functions (e.g.: scissor and grasper).

According to another aspect of this invention, these actions of switching back and forth between the different modes (e.g.: scissor and grasper) can be described as first and second extra actions.

According to yet another aspect of this invention, these extra actions are achieved without increasing the number of inputs. This is critically important for a surgical robotic arm due to the space constraints as well as compatibility requirements with legacy designs, which typically contain four inputs.

### BRIEF DESCRIPTION OF THE DRAWINGS

The invention will now be described here below with the help of drawings and diagrams provided with this application.

FIG. 1 shows the orthographic view of the entire tool in its preferred embodiment with a distal end and a proximal end. This view in FIG. 1 is from its upper left side.

FIG. 2 shows a close-up orthographic view of the distal end of the said tool's preferred embodiment with an end effector which includes a set of arms. This embodiment of the end effector may switch the configuration of the set of arms between performing a first function in a first mode of operation and a second function in a second mode of operation. In some embodiments, these functions may either be a scissor or a grasper depending upon the chosen mode of operation.

The end effector at the distal end may be arranged to the main shaft such that they may be able to pivot around two different axes, in which the axes may be on planes perpendicular to each other. These axes are shown in FIG. 3.

The end effector may also be able to rotate about the axis parallel to the main shaft of the said instrument. This axis is shown in FIG. 4.

Another combination of rotations is shown in FIG. 5.

FIG. 6 shows a preferred embodiment of the present invention with a set of arms of the end effector configured in the scissor mode of operation. The individual members of the set of arms of the end effector can be moved in a certain way in order to achieve a shearing motion necessary for the function of a scissor.

FIG. 7 shows a preferred embodiment of the set of arms of the end effector configured in the scissor mode of operation, and in an open jaw state.

FIG. 8 shows a preferred embodiment of the present invention with a set of arms of the end effector configured in the grasper mode of operation. The individual members of the set of arms of the end effector can be moved in a certain way in order to achieve a gripping motion necessary for the function of a grasper.

FIG. 9 shows a preferred embodiment of the set of arms of the end effector configured in the grasper mode of operation, and in an open jaw state.

FIG. 10 illustrates the details of the arms 004 or 006. This figure is a side view of the said arms.

FIG. 11 illustrates the details again of the arms 004 or 006, but in an orthographic view from its outer side.

FIG. 12 illustrates the details again of the arms 004 or 006, but in an orthographic view from its inner side.

FIG. 13 illustrates the details of the arms 005 or 007. This figure is a side view of the said arms.

FIG. 14 illustrates the details again of the arms 005 or 007, but in an orthographic view from its outer side.

FIG. 15 illustrates the details again of the arms 005 or 007, but in an orthographic view from its inner side.

FIG. 16 shows the arm 006 and arm 007 interlocked with each other using a certain set of locking mechanism, illustrating the scissor mode of operation. In this figure, the other two arms 005 and 004 are not visible.

The arm 006 has moved out of the locked position to arm 007, and thus is free from it. This is shown in FIG. 17. In this figure, the other two arms 005 and 004 are not visible.

FIG. 18-A shows the arms 006 and 007 corresponding to the position in FIG. 16, from the inner side view. In this figure, the other two arms 005 and 004 are not visible.

FIG. 18-B shows the arms 006 and 007 corresponding to the position in FIG. 17, from the inner side view. In this figure, the other two arms 005 and 004 are not visible.

In FIG. 19-A, the arms 006 and 005 are made visible. This illustrates the orthographic view of the position corresponding to the one shown in FIG. 17, from the lower left side of the instrument. The locking elements to hold together arms 005 and 006 are also visible.

In FIG. 19-B, the arms 006 and 005 are made visible. This illustrates the orthographic view of the position corresponding to the one shown in FIG. 17, from the upper right side of the instrument. The locking elements to hold together arms 005 and 006 are also visible.

FIG. 20 illustrates an exploded view of the said instrument's arms 005 and 006, thus making the interlocking elements between these two arms visible.

FIG. 21-A illustrates the orthographic view of the side right wall of the instrument having a characteristic set of slots which form a cam surface.

FIG. 21-B illustrates the orthographic view of the side left wall of the instrument having a characteristic set of slots which also form a cam surface.

FIG. 22 details the characteristic slots present on both the side left wall as well as the side right wall, except that on either of them, the slots are mirror images of each other. These slots together form the cam surface due to which said instrument's arms will change their configuration, as explained later.

FIG. 23 illustrates the side left wall with the arm 006 in its position such that the assembly is in the scissor mode.

FIG. 24 illustrates the side left wall with the arm 006 in its position such that the assembly is in the transition between the scissor mode, moving towards the position in the grasper mode.

FIG. 25 illustrates the side left wall with the arm 006 in its position such that the assembly is at the end of the transition to a completed grasper mode.

FIG. 26 illustrates the side left wall with the arm 006 in its position such that the assembly is in the grasper mode of operation.

FIG. 27 illustrates the side left wall with the arm 006 in its position such that the assembly is in the transition between the grasper mode, moving towards the position in the scissor mode.

FIG. 28 illustrates the side left wall with the arm 006 in its position such that the assembly is at the end of the transition to a completed scissor mode.

FIG. 29 illustrates the entire end effector of the said tool's preferred embodiment corresponding to the position shown in FIG. 25. This is the state of the end effector when it has just completed the transition to the grasper mode of operation.

FIG. 30 illustrates the entire end effector of the said tool's preferred embodiment corresponding to the position shown in FIG. 28. This is the state of the end effector when it has just completed the transition to the scissor mode of operation.

FIG. 31 illustrates from a top-front-left view the side left wall attached to the side right wall aligned through means of various concentric holes in both these walls, as well as certain protrusions and recesses in them. Also shown in FIG. 31 are pivot rods inserted at various locations.

FIG. 32 also shows the same configuration as FIG. 31, but from a top-front-right view of the said instrument.

FIG. 33-A illustrates a pulley which may be attached to a multitude of positions on the above assembly, in order to guide various wire-ropes around.

FIG. 33-B illustrates a peg stopper, which also functions to guide the wire-ropes within the pulley grooves.

FIG. 34-A illustrates a preferred embodiment of the present invention in which side left wall and side right wall, along with the pivot rods, are shown attached together to form the middle unit of the said tool, also connected to the various pulleys, around which wire-ropes will be guided.

FIG. 34-B represents the arrangement of some of the several possible set of input links, which may be wire-ropes attached to various members of the end effector. Shown in a preferred embodiment in FIG. 34-B are wire-ropes W1, W2, W3 and W4.

FIG. 34-C represents the arrangement of some more of the several possible set of input links, which may be wire-ropes attached to various members of the end effector. Shown in a preferred embodiment in FIG. 34-C are wire-ropes W5 and W6.

FIG. 35-A illustrates a preferred embodiment of the proximal end which will be coupled to the distal end of an articulated 6 axis robotic arm. Also shown in FIG. 35-A are coupling pulleys in order to connect to the robotic arm.

FIG. 35-B illustrates the internal mechanism which may consist of various pulleys for guiding the various wire-ropes, in a preferred embodiment of the proximal end of the said instrument.

FIG. 36 illustrates the free-body-diagram of the arm 006, showing forces as it slides along its slot over the central axis 017. It also shows the forces applied on the protrusion below which follows the slot. Later, we calculate the critical angle θ of slope of the curve at which the torque M applied on the arm just cancels the opposing friction forces, given coefficients of friction to be μ and μ₂ between the protrusion of the arm and curved slot and between the central axis and slot of arm. This is explained in greater detail later, with the help of description of drawings.

FIG. 37 shows the relationship between θ and μ assuming μ=μ₂. It also suggests various values of μ for various combinations of metals, with lubrication or not.

FIG. 38 shows the figure that is used derivation of the locus of a curve, which is the most efficient path to merge the two tracks of the characteristic slot shown in FIG. 22.

The actual derivation of the curve is shown in the description below.

The invention may be more fully understood by reference to the cited figures and details of exemplary embodiments. Alternative embodiments of the invention as claimed, and providing the benefits of the novel concepts of the invention, are contemplated and will be obvious from the explanations hereinafter.

### DETAILED DESCRIPTION

Minimally invasive techniques, and in particular, laparoscopic surgical methods have offered many benefits to patients. The existing laparoscopic surgical tools, however, allow the surgeon only limited flexibility of motion at the surgical worksite. The rigid shafts of most of the current laparoscopic instruments make it difficult to approach a surgical site through a small incision.

Further, various instruments have to be repeatedly removed and inserted, in effect, switched with one another in different parts of a surgery. This repeated switching action makes it difficult for the surgeon to concentrate on the procedure at hand, and also makes him or her dependent upon the scrub nurse to hand in the next required tool.

A part of the above problems can be solved by use of articulated instruments capable of wrist-like motion. Another, distinct part of these problems can be solved by the use of instruments that are capable of performing multiple functions. Both the above problems can be solved by the design of articulated instruments capable of wrist-like motion, as well as having multiple functions in the same instrument. This invention aims to create such an instrument.

Some embodiments of the present invention are described below, which make it possible for it to perform wrist-like motions, as well as the functions of a scissor and a grasper.

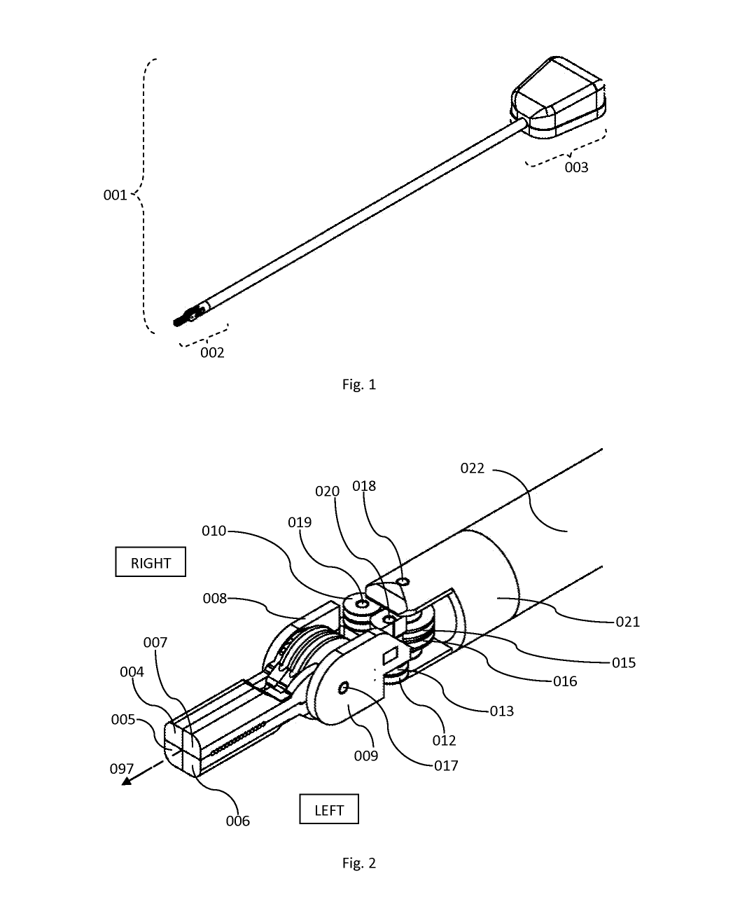

In the FIG. 1, a robotic arm with a long shaft, a distally located end effector and a proximally located robotic arm coupling as preferred embodiments of the present invention are generally indicated by the reference number 001.

The assembly indicated by 001 has a distally located end effector, generally indicated by reference number 002 as well as a robotic arm coupling, generally indicated by reference number 003.

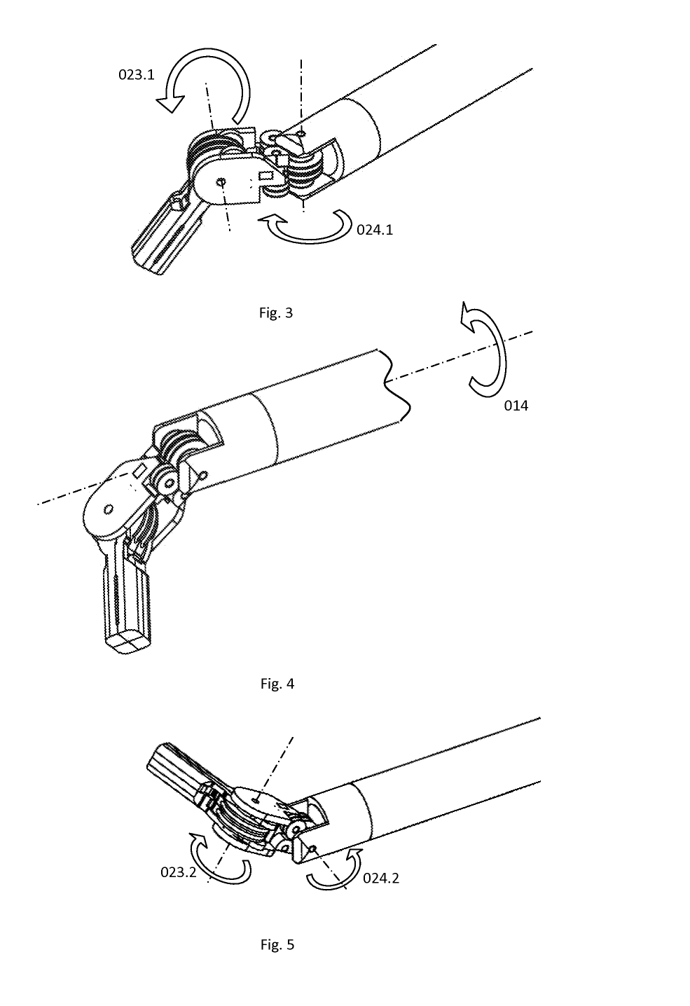

Shown in FIG. 2, is a detailed drawing of the assembly of a preferred embodiment of assembly of the end effector 002. This assembly consists of a multitude of pivot joints, which result in a wrist-like mechanism. The entire end effector is mounted at the distal end of a main bracket generally indicated by reference number 021, which itself is attached by its proximal end to the distal end of the shaft 022.

The first of such a pivot point is located at the distal end of main bracket 021. Attached to this pivot axis are the pulley sections generally indicated by reference numerals 016 and 015, of the side left wall and side right wall, generally indicated by reference numbers 009 and 008 respectively.

Another such pivot point is located at the distal end of the side left wall and side right walls, joined by a central arms axis, generally indicated by reference number 017.

In FIG. 3, the preferred embodiment of the present invention is shown in a state where it has been rotated at the first and second pivot points described above, in the directions 023.1 and 024.1 from the diagrams.

In FIG. 4, the preferred embodiment of the present invention is shown where it has been rotated from the previous position shown in FIG. 3, along the shaft 022 axis, which is generally indicated by reference number 014.

In FIG. 5, the current instrument's preferred embodiment is shown having rotated about the axes generally indicated by reference numbers 023.2 and 024.2.

These above motions are similar to that of a human wrist, thus enabling the robot to mimic actions performed by a human hand.

Referring back to FIG. 2, a multitude of arms are hinged on the axis 017. In a preferred embodiment, there are four such arms, generally indicated by reference numbers 004, 005, 006 and 007, which are capable of locking internally to each other in different combinations, resulting in different functions when they rotate about the axis 017.

A set of input links (e.g. wire ropes) attached to two of these arms transmit motion into them from the robotic arm coupling 003. These wire-ropes are routed through a multitude of pulleys mounted at characteristic locations on side left wall and side right walls. These pulleys are generally indicated by reference numerals 010, 011, 012, 013.

Due to the different interlocking mechanisms built into the set of arms, it is possible to carry out different functions using the same tool. In a preferred embodiment, the arms lock together such that in one configuration, they perform a first function in a first mode of operation (e.g. scissor). In another different configuration of this set of arms, they perform a second function in a second mode of operation (e.g. grasper). The switching back and forth between the first mode and second mode can be achieved by a first extra action and a second extra action respectively. The methods by which these functions and actions can optimally be achieved is disclosed hereunder.

In a preferred embodiment of the present invention, arms 004 and 006 move in a direction distal to the instrument, generally indicated by reference arrows 026 and 027 in FIG. 6. In many embodiments, this may be a first extra action on part of the set of arms. In this configuration, the arm 004 locks to arm 005 and at the same time, arm 006 locks to arm 007. This enables the preferred embodiment of the instrument to perform a first function in a first mode of operation (e.g. scissor).

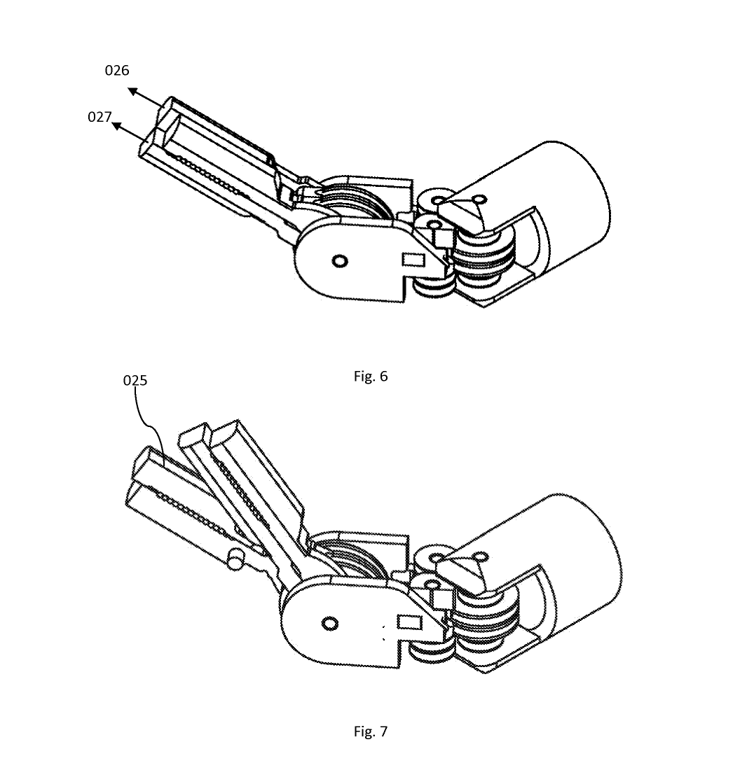

Referring to FIG. 7, the shearing edge of the arms of a preferred embodiment of this invention is displayed, generally indicated by reference number 025. The opposing shearing edge is symmetrically located on the other pair of arms, however, it is not seen in FIG. 7.

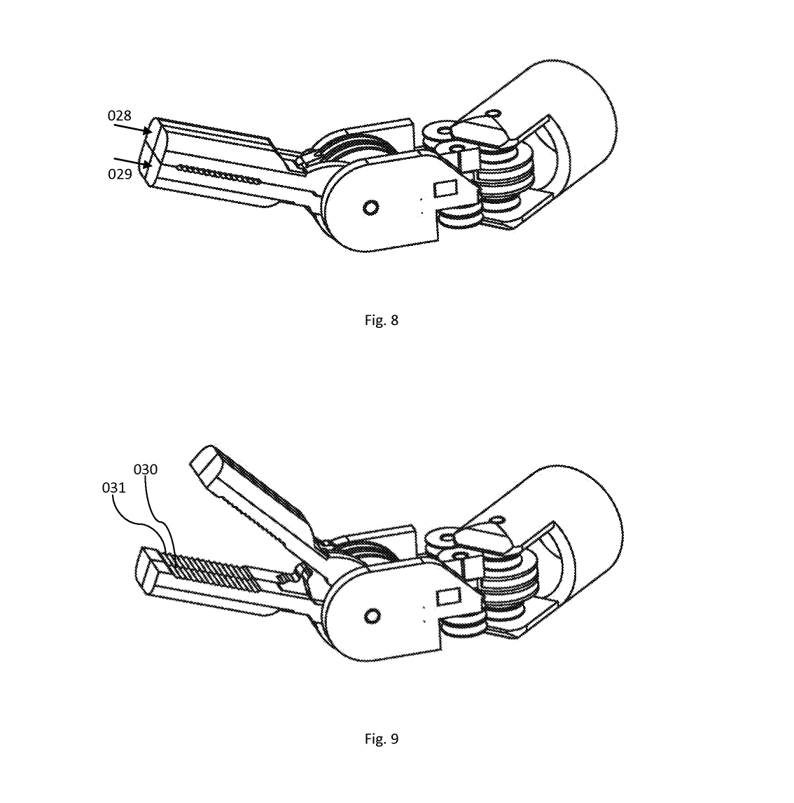

In a preferred embodiment of the present invention, arms 004 and 006 move in a direction proximal to the instrument, generally indicated by reference arrows 028 and 029 in FIG. 8. This may be a second extra action on part of the set of arms. In this configuration, the arm 004 locks to arm 007 and at the same time, arm 006 locks to arm 005. This enables the preferred embodiment of the instrument to perform a second function in a second mode of operation (e.g. grasper).

Now referring to FIG. 9, are shown one embodiment of the serrations required for the function of grasping, being generally represented by the numeral 030 and 031. A this point we must note that other embodiments of this configuration are also possible. For example, the serrations and the overall shape of the arms may be such that in this mode, the instrument functions as a needle driver. On the other hand, with sufficient insulation layers in between the arms, it is possible to use this mode as a bipolar cautery.

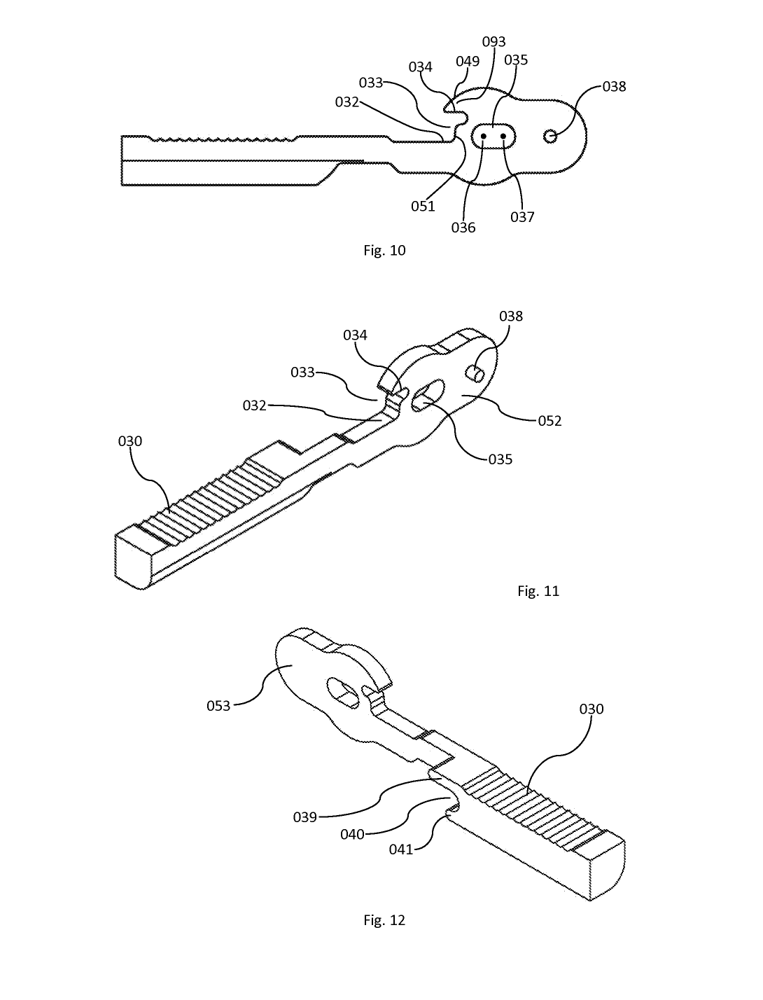

In FIG. 10 are shown details of arm 004 or arm 006, both of the same design. In a preferred embodiment, it includes a slot generally indicated by reference numeral 035, with end-points of the slot by reference numbers 036 and 037.

Again, in FIG. 10 is shown a curvature on the upper side of arm 004 or 006, generally indicated by the reference number 049, this curvature having a centre at point 036. With another flat surface, generally indicated by reference numeral 034, this curvature 049 together forms a protrusion 093.

Referring again to FIG. 10, the preferred embodiment of the arm 004 or 006 includes another horizontal surface generally indicated by reference number 032, parallel to surface 034, but lower in position. They are connected by a curved surface, generally indicated by reference number 051, which together form a depression in the part, generally indicated by reference numeral 033.

Referring to FIG. 11, a protrusion at the proximal end of the arm 004 or 006 is shown as the preferred embodiment, generally indicated by reference number 038. By moving along a specially constructed track which will be explained later, this protrusion, which may be regarded as a cam follower, guides the motion of the arm 004 or 006. This track motion is also responsible for the tool functioning as a scissor or as a grasper or the switches between the two modes of operation.

Also shown in FIG. 11 are the serrations 030. Also shown in FIG. 11 is the outer face of the arm 004 or 006, generally indicated by reference number 052.

Shown in FIG. 12 is the arm 004 of or 006, which typically includes another depression, generally indicated by reference number 040. This depression is created in between two mechanical support members indicated by 039 and 041. Also shown in FIG. 12 is the inner face of arm 004 or 006, indicated by reference 053.

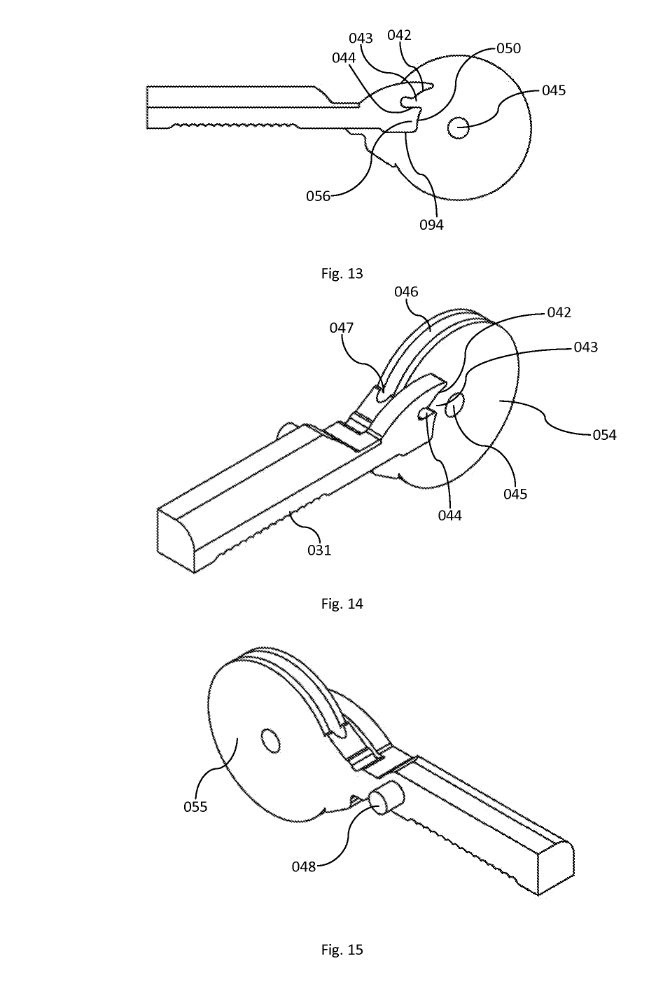

Referring to FIG. 13, what is shown is the preferred embodiment of the arm 005 or 007, from its outer side view. This arm 005 or 007 typically includes a hole for pivot axis which is generally indicated by reference number 045.

Around the hole is typically a pulley, which is indicated by reference number 046, which is shown in FIG. 14.

To clamp down a wire rope drawn over the pulley 046, a mechanism is placed at its distal end, generally indicated by reference number 047, also illustrated in FIG. 14. This mechanism may be a mechanical clamp, friction, or other means to fasten the wire-rope to the pulley.

Referring back to FIG. 13, the arm 005 or 007 typically includes a curved surface, generally indicated by reference number 042. This surface has its center of curvature at a point that is located in the distal direction, a certain distance from the center of the hole 045 which is equivalent to the distance between the points 036 and 037 of arm 004 or 006.

The arms 005 or 007 also include two horizontal surfaces generally indicated by reference numbers 044 and 094. These above surfaces create the required depressions and protrusions in the arms, which are necessary to create the inter-locking of the arms in different configurations to yield various functions, such as the scissor and grasper in a preferred embodiment. A curved surface generally indicated by reference numeral 050 connects the proximal ends of the surfaces 044 and 094.

In a preferred embodiment of the present invention, one depression is created by the two surfaces 042 and 044, which is generally indicated by reference number 043. A protrusion is also included in the preferred embodiment, generally indicated by reference number 056.

Referring to FIG. 14, the arm 005 or 007 also includes as the preferred embodiment of the invention, serrations for the grasping action of the tool, which are generally indicated by reference numeral 031. FIG. 14 also shows an outer surface of the pulley 046, generally indicated by reference number 054.

Referring now to FIG. 15, is shown the inner side orthographic view of the arm 005 or 007 with some details, including a protrusion generally indicated by reference numeral 048. FIG. 15 also shows the inner surface of pulley 046, which is generally indicated by reference number 055.

In a preferred embodiment of the present invention, the scissor mode of operation occurs when the arms 006 and 007 lock to each other on one hand, while on the other, arms 004 and 005 lock to each other.

Such an arrangement is illustrated in FIG. 16, which shows arm 006 locked to arm 007. This occurs due to the inter-locking members on either arm locking into each other. In a preferred mode of this action, the protrusion 093 from arm 006 locks into the depression 043 of arm 007. At the same time, the protrusion 056 of arm 007 also locks into depression 033 of arm 006. In this configuration, the arm 006 has moved in such a position, that along the axis 017 common to all four arms, the point 036 of arm 006 aligns with the centre of the axis, as also the centre of hole 045.

Referring back to FIG. 7, the preferred embodiment of the present invention is illustrated in the scissor mode, with all the inter-locking mechanisms in position as described above. This may be regarded as the end effector being configured to perform a first function in a first mode of operation. In this configuration, the cutting edge 025 acts to shear the required object that the user of this instrument intends to cut. Typically, such an object would be a piece of flesh, internal organ, vessels, tissue, suture, etc. during surgery.

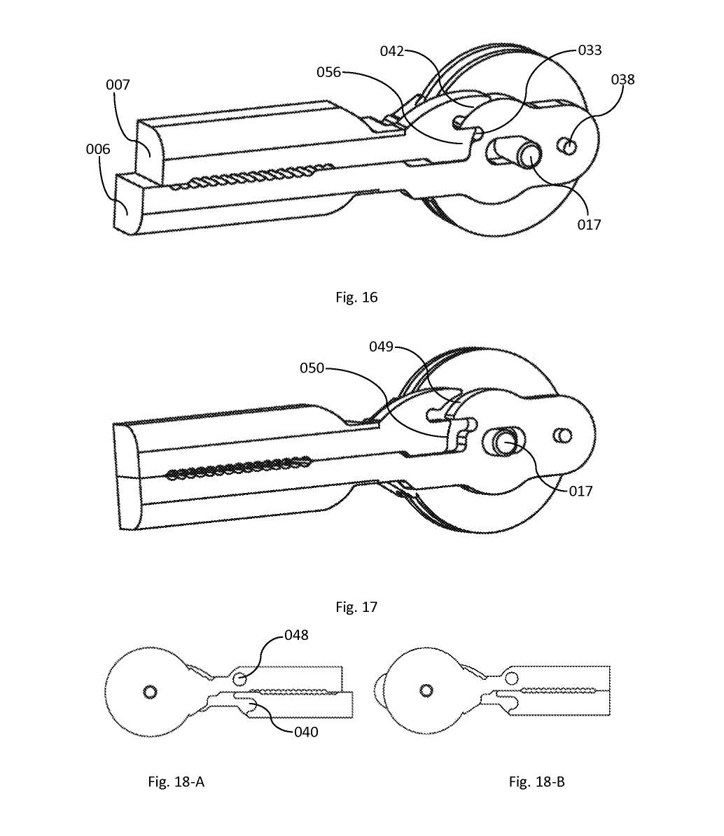

The inner side of arms 006 and 007 are shown in FIG. 18-A. They indicate the position of protrusion 048 of arm 007 and the depression 040 on arm 006 in the scissor mode.

Referring to the FIG. 17, the preferred embodiment of the present invention is illustrated such that arm 006 and arm 007 are in positions that correspond to the grasper mode. This may be regarded as the set of arms of the end effector being configured to perform a second function in a second mode of operation. In this configuration, the previously locked positions of the protrusions and depressions during the scissor mode are no longer in the locked state, but in fact, free to rotate about the central axis through point 037 on arm 006 or the hole 045 on arm 007. Thus, the protrusion 093 of arm 006 is no longer locked in the depression 043 of arm 007, while at the same time, the protrusion 056 of arm 007 is no longer locked in the depression 033 of arm 006.

During the grasper mode of the preferred embodiment of the invention, the arms 004 and 007 lock to each other, and at the same time, the arms 005 and 006 lock to each other. This is because the protrusion 048 of arm 007 locks into the depression 040 of arm 004, while at the same time, the protrusion 048 of arm 005 locks into the depression 040 of arm 006.

The relative positions of the protrusion 048 of arm 007 and depression 040 of arm 006 in this grasper mode, are illustrated from the inner view in FIG. 18-B.

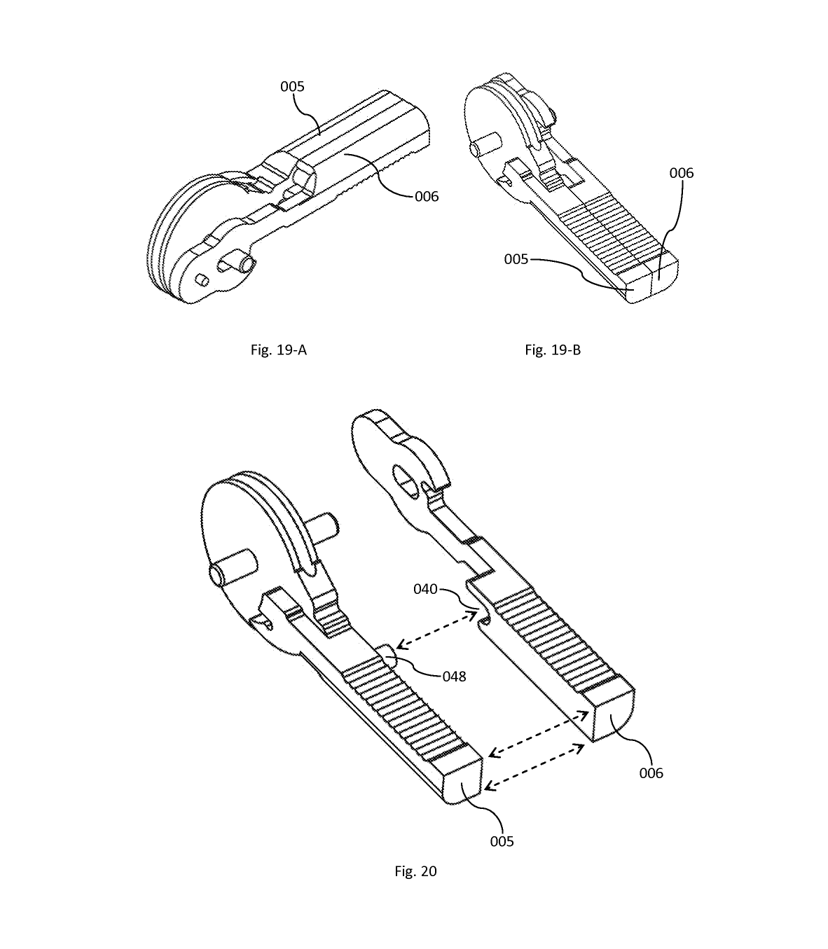

The locking mechanism between arms 005 and 006 through the protrusion 048 and depression 040 respectively, is further illustrated in FIG. 19-A, from a lower left angle. FIG. 19-B also illustrates the same mode of grasper configuration of arms 006 and 005 but from the top right angle. FIG. 20 shows an exploded view of arms 005 and 006, moved away from each other, to better illustrate the corresponding protrusion 048 and depression 040.

In the grasper configuration, the arm 006 and 004 have moved in such a position as shown by arrows 029 and 028 respectively, in FIG. 8, that the point 037 of is aligned with the central axis 017.

Since the arms 004 and 007 can together rotate about the axis 017, away from or towards the arms 005 and 006, which in, turn can independently rotate about the same axis 017 away from or towards the former, the resulting motion allows the tool to function as a grasper.

In the preferred embodiment of this invention, the arms 007 and 005 are each connected by a wire-rope drawn over their own pulleys 046. These wire-ropes cause the movement of arms 007 and 005 independently.

In each case, of scissor configuration or grasper configuration, the arms 004 and 006 move accordingly since they are connected to either arm 005 or 007 depending upon the selected configuration. Thus, when in scissor mode of operation, the arm 004 is connected to arm 005 due to the above described protrusions and depressions in them mating, and hence, when arm 005 moves, arm 004 moves along with it. Similarly, in the scissor configuration, arm 006 is locked to arm 007, again by a similar protrusion and depression mechanism described above, and hence when arm 007 is moved by the wire-ropes, so does the arm 006 move with it. While, on the other hand, when the said tool is in the grasper mode of operation, the arm 004 is connected to arm 007 by a different set of protrusion and depression described above, and hence when arm 007 moves due to the wire-ropes, the arm 004 is forced to move along with it. Similarly, arm 005 is attached to arm 006 while in grasper mode, by a set of protrusion and depression, and hence, when arm 005 is moved due to the wire-rope attached to it, arm 006 moves along with it.

Thus, above it is described how the set of arms of the end effector are capable of being configured to perform a first function in a first mode of operation (e.g. scissor mode) and a second function in a second mode of operation (e.g. grasper mode) by selective locking of the set of arms to each other in different ways. Now it will be disclosed how these arms are controlled and how the switching mechanism works.

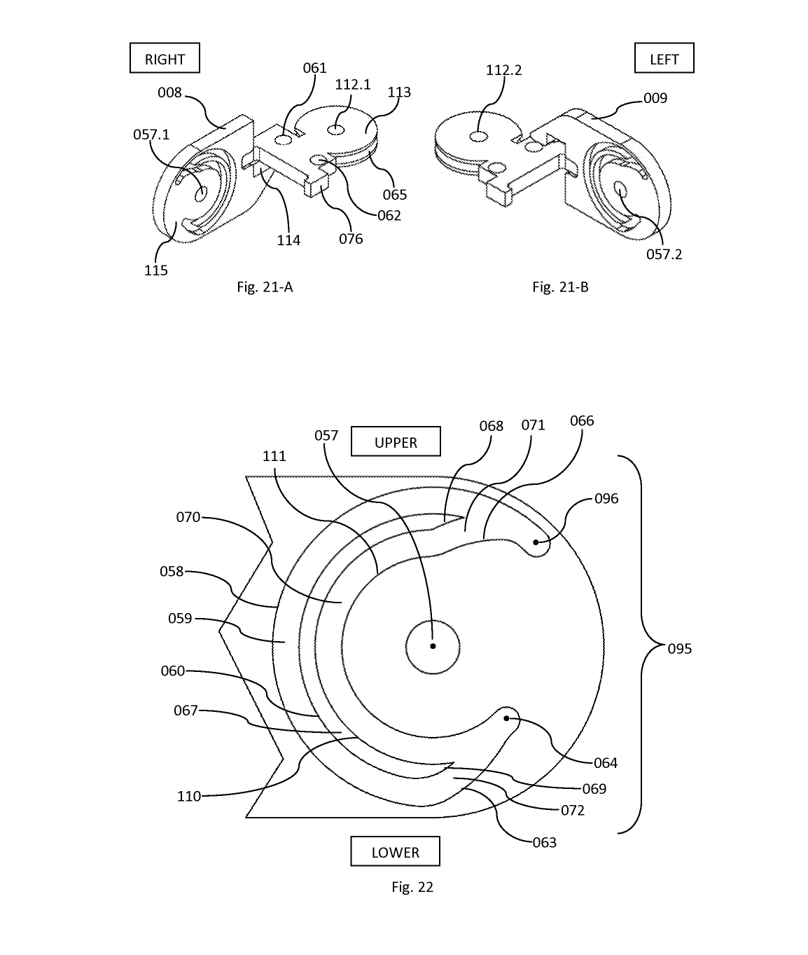

These arms are all pivoted along a single axis 017, as illustrated in FIG. 2. Also included in the preferred embodiment of the invention, and shown in FIG. 2 where they are generally represented by reference number 008 and 009 are side right wall and side left wall respectively. These are shown in greater detail in FIG. 21-A and FIG. 21-B respectively. It is between the inner faces of these side walls that the four arms are pivoted on a common axis 017. The inner faces of each of the side walls are generally represented by reference number 115.

In a preferred embodiment of the invention, each of the inner walls of the side left wall 009 and side right wall 008, have a certain characteristic set of slots which are generally represented by the reference numeral 095, in FIG. 22. These slots 095 can also be seen on the inner faces of side left wall and side right wall in FIG. 21-B and FIG. 21-A. These slots form a cam surface over which the set of arms are rotated in various portions of range of motion. For example, in a first track segment of the slot 095, the arms are rotated over a first portion of range of motion. Similarly, in second and third track segments of the slot 095, the arms are rotated over second and third portions of range of motion respectively.

Each side wall includes a hole, generally indicated by reference number 057.1 or 057.2 for right or left sides. Similarly other features are also indicated by various other reference numbers, such as 112.1 and 112.2 for side right wall and side left wall components. The reference numeral 112 generally indicates the hole at the proximal half of the side walls, through which in the preferred embodiment, passes a first pivot axis 018. Around this hole is a pulley mechanism generally represented by reference numeral 065.

Two more holes are shown on the side walls which are generally indicated by reference numbers 061 and 062. Pulley axes 019 and 020 pass through these holes and are used to mount the pulleys 010 and 011 on axis 019 and pulleys 012 and 013 on axis 020. This arrangement is shown in FIG. 2.

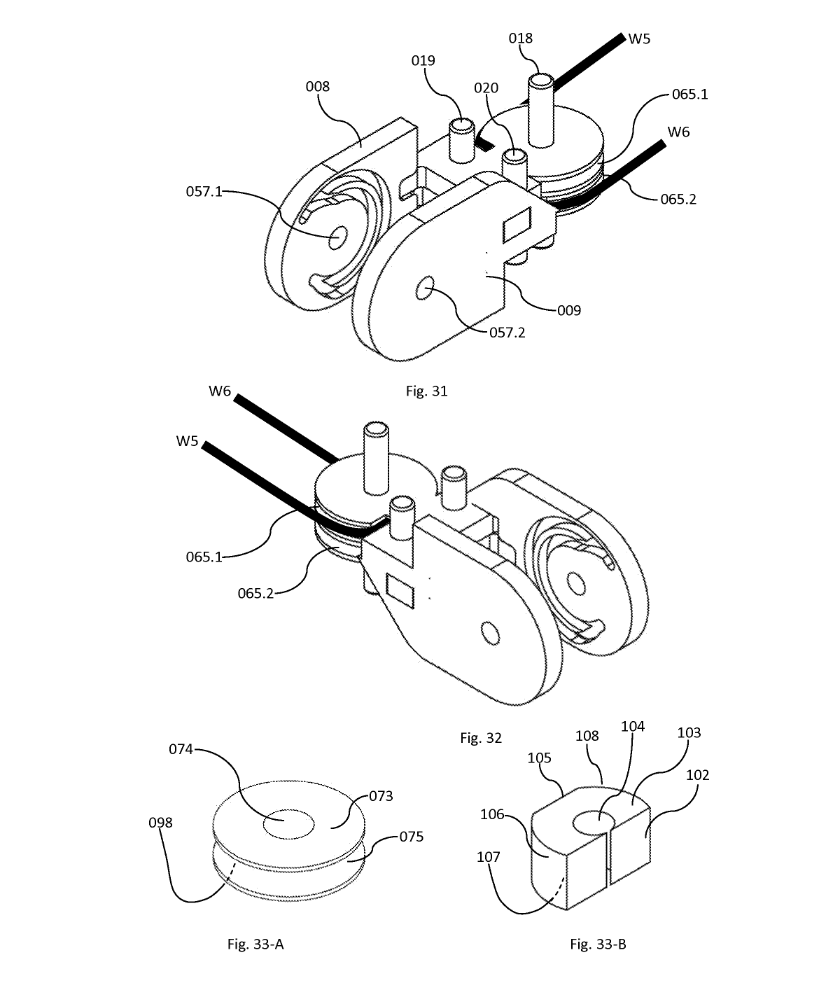

Referring to FIG. 21-A and FIG. 21-B, side walls 008 and 009 each also include a depression generally indicated by reference number 114 and a protrusion generally indicated by reference number 076. Shown in FIG. 31 and FIG. 32 is the assembly of parts 008 and 009, where the protrusion 076 of one side locks into the depression 114 of the other, thus ensuring the alignment of the two parts. Additionally, pulley axes 019 and 020, which pass through holes 061 and 062 of each of 008 and 009, also act as dowel pins, thus helping make the alignment more robust.

At this point, in the preferred embodiment are shown two axes about which the end effector may be rotated about, thus yielding a wrist-like motion for the end effector. These two axes may be in planes perpendicular to each other. When the position of the set of arms of end effector is such that the axis 017 is perpendicular to the direction of the axis of shaft 022, and any of the arms 004, 005, 006 or 007 are in a position about axis 017 such that they point in a direction parallel to the axis of the shaft 022, we get the direction 097, which gets defined as the “zero position” of the arms with respect to axis 017. This entire arrangement is illustrated in FIG. 2.

Referring back to FIG. 22, as the preferred embodiment of the present invention, a characteristic slot 095 is illustrated, which is embedded onto the inner face 115 of 008 and 009. The slot 095 collectively forms a cam surface which effects certain characteristic movements of the set of arms. The slot 095 includes several track segments of cam surfaces, including two partially circular concentric track segments, around the centre of the hole 057. A first track segment of the cam surface which is formed by the outer track, is generally represented by reference number 059, while a third track segment of cam surface is formed by the inner track, which is generally indicated by reference numeral 070. These correspond to a first portion of range of motion of the set of arms about the hinge, effected by the set of input links. These tracks are smoothly joined to each other at either ends via characteristic curves. They form a second and fourth track segment of cam surfaces represented by reference number 071 on the upper side, and the reference number 072 on the lower side. The tracks, after joining each other, end at points generally represented by reference numbers 096 and 064 at the upper and lower ends respectively. These tracks, as members of the slot 095 are in essence the cam surfaces that cause movements and hence changes in the configuration of the arms. When the protrusion 038, which may be a follower, follows the upper and lower connecting tracks, the set of arms rotate through a second and a third portion of range of motion effected by the set of input links. A more detailed explanation of these motions is explained subsequently.

The outer track 059 includes an inner face and an outer face, generally indicated by reference numbers 060 and 058 respectively. Similarly, the inner track 070 includes an inner face and an outer face, each generally indicated by reference numbers 111 and 110 respectively. Separating the inner and outer tracks is a central wall, generally indicated by reference numeral 067. This wall is generally enclosed by surfaces 060 and 110.

On the upper side, the inner track 070 leaves its own path to smoothly merge outwards to outer track 059, via the upper connecting track 071. This upper connecting track 071 includes an inner face and an outer face, generally represented by reference numbers 066 and 068 respectively.

Similarly, on the lower side, the outer track 059 leaves its own path to smoothly merge inwards to inner track 070, via the lower connecting track 072. This lower connecting track 072 includes an inner face and an outer face, generally represented by reference number 069 and 063 respectively.

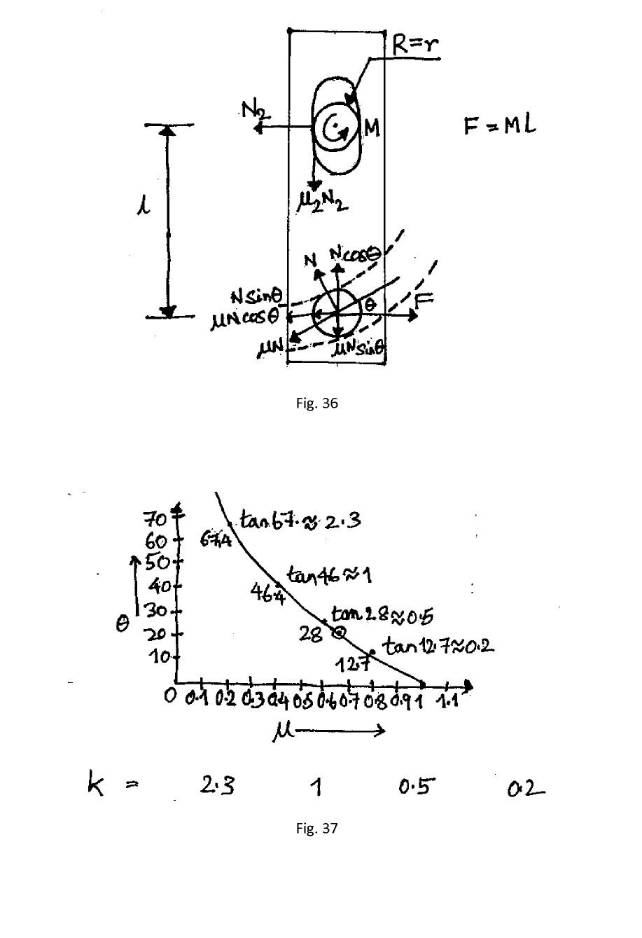

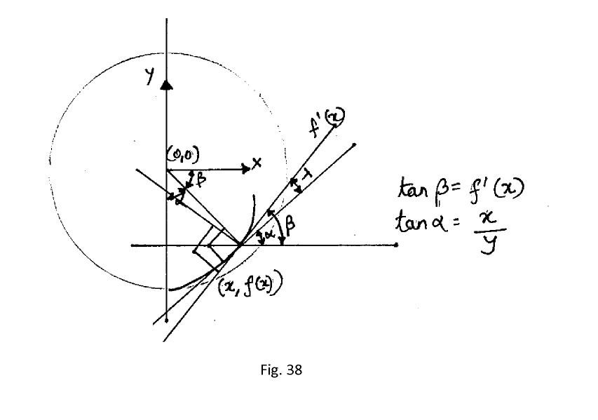

These upper connecting track 071 and lower connecting track 072 respectively terminate at end-points 096 and 064 respectively. The characteristic curvature of these tracks 071 and 072 is explained later in this description, with the help of FIG. 36, FIG. 37 and FIG. 38.

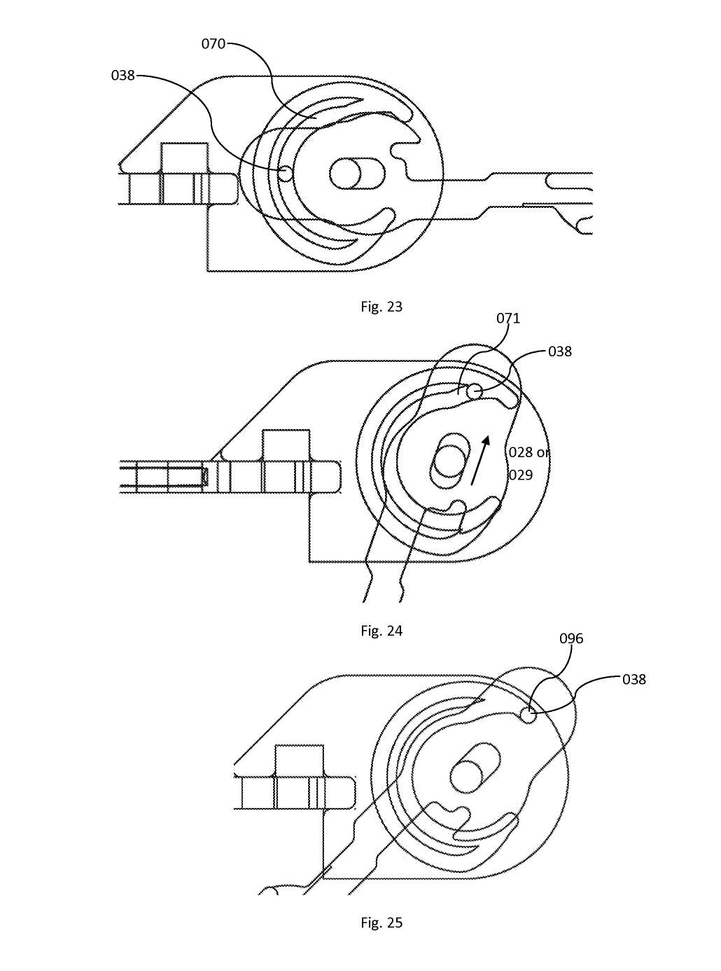

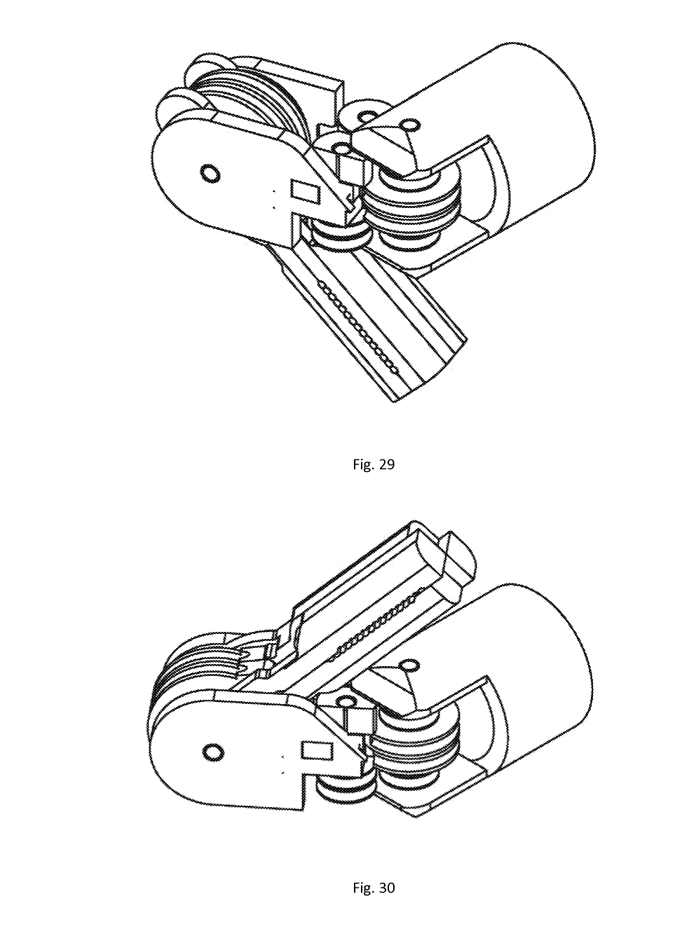

Referring to FIG. 23 to FIG. 28, the inner side of side left wall 009 along with the arm 006 at various positions is shown. The face 052 of arm 006 is parallel to the face 115 of side left wall 009. The hole 057 aligns with the axis 017 concentrically, as well as with a particular point on the slot 035 between points 036 and 037, depending upon the mode of operation of the instrument. The protrusion 038 of arm 006 follows the slot 095 of side left wall 009 as the arm 006 rotates about either points 036 or 037 on the axis 017, or the slot 035 slides along the axis 017 while the instrument is in a state of transition between its modes of operation. In this explanation above and subsequent description below, arm 006 and side left wall 009 are referred, however, a similar arrangement and function follows correspondingly for arm 004 and side right wall 008.

In FIG. 23, the protrusion 038 of arm 006 is a follower and is shown to be following the inner track 070 of the characteristically curved slot 095. This arrangement corresponds to the instrument being in the scissor mode of operation. In this position, the arms 006 and 007 are locked to each other, as described above, and illustrated in FIG. 16, FIG. 6 and FIG. 7. Similarly, the arm 004 is locked to arm 005. Arm 005 and 007 are capable of being rotated about axis 017 independently of each other, using a set of input links, (e.g. wire-ropes). As arm 005 and arm 007 are each connected respectively to arm 004 and arm 006, the latter are also forced to rotate accordingly about axis 017. In this configuration, the arms 006 and 004 rotate about the point 037. In the preferred embodiment of the present invention, as the set of arms rotate in either clockwise or counter-clockwise directions, but are within an angle of 90° of the direction 097, the protrusion 038 follows the inner track 070, thus preventing the arms 006 and 007 from sliding along their slot 035 over the axis 017. This rotation of the set of arms may be regarded as being carried out in a first portion of range of motion.

In FIG. 24, the arm 006 is being shown in a state of transition from being in the scissor mode of operation to the grasper mode of operation. This position is arrived at when the arm 006 rotates about axis 017 an angle greater than 90° in the clock-wise direction when viewed as shown in FIG. 23 and FIG. 24. As the protrusion 038 leaves inner track 070 and enters the upper connecting track 071, it is pushed outward, to depart from its original circular course of movement by coming in contact with the surface 066 of the connecting track 071. Due to this push, and subsequent movement that results, the arm 006 begins to move in a direction generally indicated by arrow 028 or 029. Thus, the arm 006 begins to slide along its slot 035, from a position where initially the axis 017 aligned with point 037, towards another position, where the axis 017 would align with point 036. This results in a change of configuration of the set of arms of the end effector, from performing a first function in a first mode of operation, to performing a second function in a second mode of operation. This rotation of the set of arms may be regarded as being carried out in a second portion of range of motion around the hinge, effected by the set of input links.

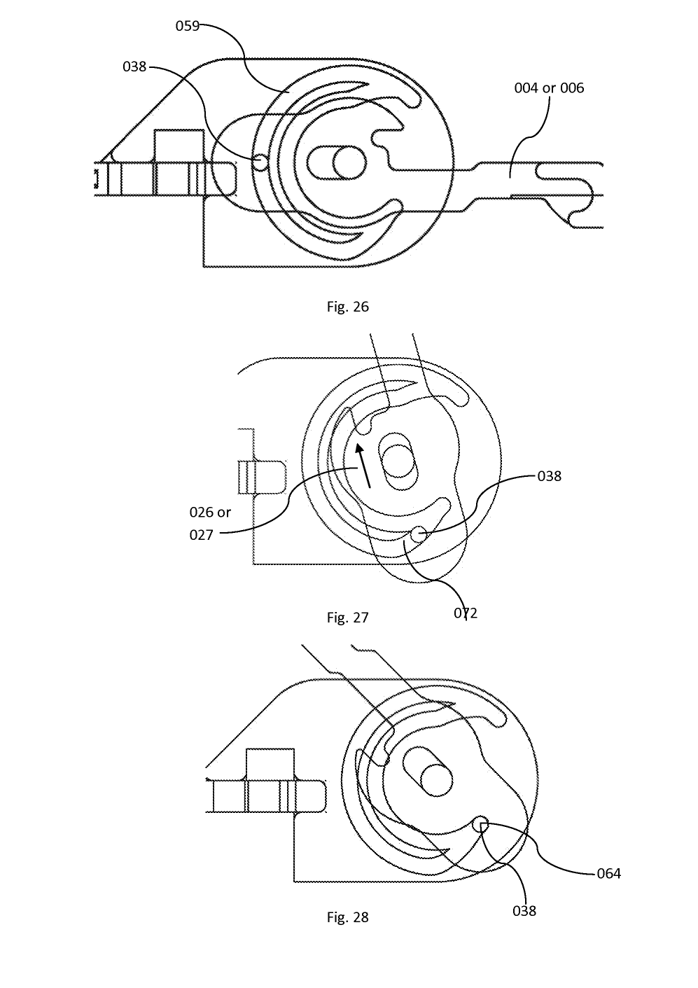

As the arm 006 rotates further along, the protrusion 038 follows the upper connecting track 071 all the way till it reaches and coincides with point 096. At this position, the arm 006 has also slid across axis 017 along its slot 035, with the axis 017 coinciding with point 036. This results in the completion of the transition from scissor to grasper mode of operation of the instrument and is illustrated in FIG. 25, showing only arm 006 and side left wall 009. The same configuration is illustrated in FIG. 29, showing the entire end effector in its preferred embodiment.

Thus, in this position, the arm 006 is no longer locked with arm 007, but instead gets locked with arm 005. At the same time, arm 004 is no longer locked with arm 005, but is now locked with arm 007. When arms 005 and 007 are rotated about axis 017, by means of the set of input links (e.g. wire-ropes), they also force arms 006 and 004 respectively to rotate along with them, with the resultant motion rendering the function of a grasper in this preferred embodiment of the end effector.

When the arm 006 rotates back counter-clockwise along axis 017 to the central direction 097, it results in the protrusion 038 following a path of track 059. Also, the arm 006 rotates about its point 036 on the axis 017. This position is illustrated in FIG. 26, with only side left wall 009 and arm 006 visible. A similar position is also illustrated in FIG. 8, where the arms are at a different angle than the one pointing parallel to 097.

Since the arms 005 and 007 are capable of rotating independently of each other, due to the wire ropes attached to them, they may rotate away from or towards each other, also forcing along with them arms 006 and 004 respectively, thus resulting in a motion which corresponds to the opening or closing respectively of the jaws of the grasper. These positions are illustrated in FIG. 8 and FIG. 9.

As the arm 006 rotates further counter-clockwise about the axis 017, and reaches angles greater than 90°, the protrusion 038 of arm 006 follows the path of the slot 095 from the outer track 059 to the lower connecting track 072, and begins moving towards the inner track. As this motion occurs, the surface 063 of the lower connecting track 072 exerts an inward force on the protrusion 038 that is following it. As a result, the arm 006 is simultaneously forced to slide along its slot 035, from a position where the point 036 had aligned with the axis 017 to a position where the axis 017 aligns with a point in between points 036 and 037. This is the position of the preferred embodiment of this invention where it is in a state of transition from the grasper mode back to the scissor mode. This is the position when the set of arms are again changing their configuration from performing the second function in a second mode of operation, back to performing the first function in the first mode of operation. This motion may be regarded as being carried out while the set of arms are being rotated in a third portion of range of motion by the set of input links.

As the arm 006 continues its counter-clockwise rotation about axis 017, the protrusion 038 follows the lower connecting track 072 all the way to point 064. At this position, the arm 006 has slid all the way along slot 035 from the point 036 being aligned with axis 017 to now the point 037 being newly aligned with the axis 017. This resulting position corresponds to the state of the preferred embodiment of the present invention where the transition from the tool being in a grasper mode to the tool being in the scissor mode is complete. As described earlier, in this configuration, the arm 006 is no longer locked to arm 005, but instead gets locked to arm 007. At the same time, arm 004 is no longer locked to arm 007, but is now locked to arm 005 instead. This position is illustrated in FIG. 28, where only the arm 006 and side left wall 009 are visible. It is also illustrated in FIG. 30, where the entire end effector of the preferred embodiment is shown.

As the arms 005 and 007 can rotate independently of each other via the wire-ropes attached to them, they may move away from or towards each other, also forcing along with them arms 004 and 006 respectively, and resulting in the opening or closing respectively of the jaws of the scissor. This is illustrated in FIG. 7.

Why this particular characteristic curvature of the upper connecting track 071 and lower connecting track 072 is of special importance in the preferred embodiment will be explained later, with help of FIGS. 36 to 38.

Referring to FIG. 31 now, the assembly of side left wall 009, side right wall 008, and axes 018, 019 and 020 is illustrated. The axis 018 in the preferred embodiment forms the first of pivots of the end effector, allowing relative rotation between the middle section consisting of side right wall 008 and side left wall 009 with respect to the main bracket 021, which in turn is rigidly connected to shaft 022.

FIG. 32 illustrates the same arrangement as that in FIG. 31, but from a view from the upper right side, with respect to the tool.

In the preferred embodiment of the present invention, towards the inner niche of the pulleys 065.1 and 065.2, are attached to a set of input links such as two wire-ropes, generally indicated by references W6 and W5 respectively. These wire-ropes undertake the function of rotating the entire assembly including and distal to the side left wall and side right wall, about the pivot axis 018. Thus, these wire ropes W5 and W6 fulfill one of the wrist articulation functions of the instrument. This is illustrated in FIG. 31 and FIG. 32.

Referring to FIG. 33-A, a standard side pulley is illustrated. It includes a central hole, generally indicated by reference numeral 074. It also includes a pulley edge, generally indicated by reference number 075. It has an upper surface and a lower surface, generally indicated by reference numbers 073 and 098.

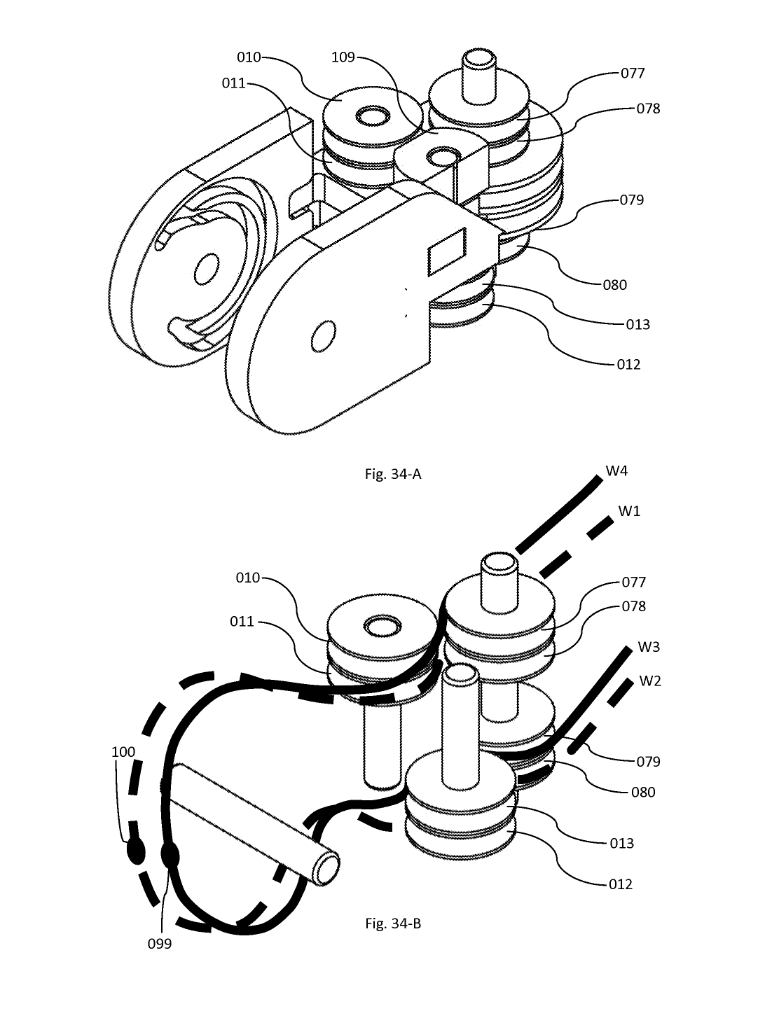

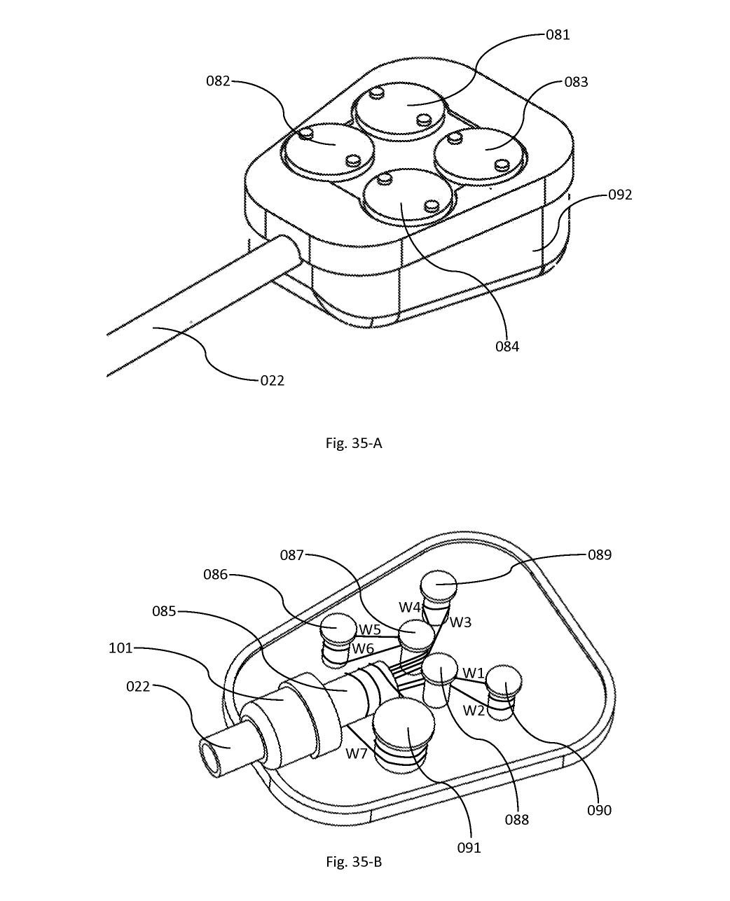

Several instances of the pulley illustrated in FIG. 33-A are used in the preferred embodiment of the invention, the assembly of which is shown in FIG. 34-A. Two pulleys, generally referred to by numbers 010 and 011, are mounted on the axis 019, above the surface 113 of side right wall.

In FIG. 33-B is shown the peg stopper, in the preferred embodiment. It includes a flat face generally indicated by the reference number 102. It also includes a top face and bottom face, generally indicated by reference numbers 103 and 107 respectively. It also further includes a curved surface, a flat face in between and another curved surface on the other side, generally indicated by reference numbers 106, 105 and 108 respectively. For fitting the peg stopper to the main assembly at either pivot axis 019 or 020, a central hole is provided, which is generally referenced by the number 104 in FIG. 33-B. The function of the surfaces 106, 105 and 108 is to provide guidance to the wire-rope onto the pulley, and prevent it from slipping out of the pulley's groove. The flat face 102 ensures the alignment of the peg stopper with respect to the side right wall or side left wall, while at the same time allowing a small amount of movement for adjustment of the wire-ropes.

There are two instances of the peg stopper in the preferred embodiment, illustrated in FIG. 34-A. Also shown in this figure is the arrangement of pulleys and peg stoppers and various axes on the side left wall and side right wall. Pivot axis 018 passes through the holes 112.1 and 112.2 of side right wall and side left wall respectively, also including on its upper side pulleys 077 and 078, and on its lower side pulleys 079 and 080. This entire arrangement is later connected to the main bracket at either ends of the pivot axis 018. The axis 019 passes through the hole 061.1 of the side right wall, hole 062.2 of the side left wall, and also includes pulleys 010 and 011 on the upper side, i.e. the one facing the surface 113, and also includes the peg stopper on the lower side (not shown). The axis 020 passes through the hole 061.2 of the side left wall, hole 062.1 of the side right wall, and also includes pulleys 012 and 013 on the lower side, i.e. the one facing the surface 113, and also includes the peg stopper on the upper side, generally indicated by reference numeral 109.

Illustrated in FIG. 34-B is the arrangement of a set of input links, (e.g. wire-ropes) which are drawn over the arms 005 and 007, in order to execute their rotation about axis 017. In the preferred embodiment of this invention, a wire rope, generally indicated by the reference numeral W1 is drawn from the outer side of the pulley 078, and moving over the inner side of pulley 011, eventually rides over the groove of the pulley of arm 005, and ends at the stem of arm 005. Here, it is joined to the next part of the wire-rope at a joint generally indicated by reference number 100. The next part of the wire rope is generally indicate by reference number W2, now rides over the lower half of the pulley of arm 005 and winds around the inner side of pulley 012, followed by winding around the outer side of pulley 080, before eventually exiting the end effector mechanism, and getting drawn into the shaft. When wire-rope W1 is pulled, the wire-rope W2 is let go at the same rate, thus rotating the arm 005 in the upper direction. When the wire rope W2 is pulled and wire rope W1 let go at the same rate, the arm 005 rotates in the downward direction. Another wire-rope, generally indicated by reference number W4 enters from the top half, winds around the pulley 077 on its outer side, followed by winding around the pulley 010 on its inner side, finally going over the pulley of arm 007, and ending at the stem of the arm 007. Here, it joins another section of a wire rope by a joint which is generally referred to by reference number 099. The next section of the wire-rope, generally indicated by reference number W3 winds around the bottom part of the pulley of arm 007, then winds around the inner side of pulley 013, eventually winding around the outer side of pulley 080, before exiting the end effector into the shaft 022. When wire rope W4 is pulled and wire rope W3 is let go at the same rate, the arm 007 rotates upwards about the axis 017. Similarly, when the wire rope W3 is pulled and the wire rope W4 is let go at the same rate, the arm 007 is forced to rotate downwards about the axis 017. Due to these motions of the wire ropes, the arms 005 and 007 move, in turn making the arm 004 and 006 also move when they are in different modes such as scissor or grasper.

In another embodiment of the present invention, the set of input links (e.g. wire-ropes) W1 and W2 may be a single wire-rope, attached to the stem of arm 005, and set of input links (e.g. wire ropes) W3 and W4 may also be a single rope, attached to the stem of arm 007.

The FIG. 35-A shows the coupling 003 at the proximal end of the entire tool in the preferred embodiment, where it couples to the robotic arm. The outer cover and frame of this coupling is generally indicated by reference numeral 092. It includes several pulley-connectors which are generally indicated by reference numbers 081, 082, 083 and 084 in the FIG. 35-A. Also shown is the proximal end of shaft 022, which connects to the coupling 003.

Now referring to FIG. 35-B, an opened up view of the coupling 003 is illustrated. The shaft 022 is seen attached to the distal end of the coupling. In the preferred embodiment of the present invention, the shaft is connected to the main housing of the coupling by means of a bearing, which is generally indicated by reference number 101. A barrel rises in the proximal direction from the proximal end of the shaft 022 further from the bearing 101. This barrel is generally indicated by reference number 085. Wound around this barrel 085 is another wire-rope W7, which connects to one of the pulley-connectors' inner spindle. This spool, in one embodiment is represented by the reference number 091.

There are two more spools or pulley arrays situated proximal to the main barrel. These are generally indicated by reference numerals 087 and 088. Wire ropes W3, W4, W5 and W6 exit from inside the barrel, and wind around the pulley-array 087, finally reaching their respective spindles, generally represented by reference number 089 and 086. Wire ropes W3 and W4 terminate around the spindle 089, whereas the wire-ropes W5 and W6 terminate around the spindle 086. At the same time, wire-ropes W1 and W2 wind around the spindle 088, finally terminating at the inner-spindle generally indicated by reference number 090.

The inner spindles 086, 089, 090, 091 are directly attached to the pulley-connectors 084, 083, 081 and 082. Thus, when the pulley-connectors rotate when attached to the robotic arm, the inner spindles also rotate, thus moving the wire-ropes W1 to W7 as controlled by the robot. These wire-ropes in turn enable the movement of the set of arms of the end effector, as well as its various degrees of freedom, to achieve the wrist-like articulated motion.

Referring now to FIG. 36, it is a free-body-diagram generally indicating the axis 017 at the top, the arm 004 or 006 along the length, the protrusion 038 at the bottom, which is shown in some parts of the slot 095. These parts may include tracks 071 or 072. A moment M is applied to the arm 004 or 006 as shown in FIG. 36. This moment reflects a force F equal to M×1 at the protrusion 038, which gets exerted on the surface or walls of the slot 095. The slot 095 in turn applies a normal reaction N on the protrusion 038. This force N is responsible for making the arm 004 or 006 slide along its slot 035. It also results in the normal reaction N₂ by the axis 017 on the surface of slot 035. These forces also give rise to frictional forces characterized by coefficients of friction μ between the protrusion 038 and surface of slot 095, and μ₂ at the pivot axis 017 and boundary surface of slot 035. The slot is shaped such that at any point, the normal to its slope makes a certain angle θ, to the line joining the point and center of the axis 017. In the equations, we equate the force exerted by the moment M, to the opposing forces resulting from friction, to calculate the critical angle θ, at which they just cancel each other. This calculation is displayed below:

$$F = N sin θ + μ N cos θ = ML \\ Upward force = N cos θ - μ N sin θ \\ {N}_{2} = N sin θ + μ N cos θ \\ ∴ For equilibrium \text{/} movement \\ \frac{N / cos θ}{cos θ} = \frac{μ N / sin θ}{cos θ} + {μ}_{2} ( \frac{N / sin θ}{cos θ} + \frac{μ N / cos θ}{cos θ} ) \\ 1 = μ tan θ + {μ}_{2} tan θ + μ \cdot {μ}_{2} \\ tan θ = ( \frac{1 - {μ}_{1} {μ}_{2}}{{μ}_{1} + {μ}_{2}} ) \qquad [ Math . 1 ]$$

It calculates the critical angle θ of slope of the curve at which the torque M applied on the arm just cancels the opposing friction forces, given coefficients of friction to be μ and μ₂ between the protrusion of the arm and curved slot and between the central axis and slot of arm.

Thus, the above explained mechanism would work when the slot 095 is constructed of any curve in which at any given point the angle its slope's normal makes to the line joining the center is less than this critical angle θ. If we assume μ=μ₂, then the resulting equation is plotted in FIG. 37.

With the help of FIG. 38, a differential equation is derived, which corresponds to the locus of a curve such that at any given point on it, the normal of the slope at that point is always at a constant angle λ to the line joining that point and the origin. The equation is subsequently solved to yield the final solution equation. This derivation is as follows:

$$β - ∝ = constant \\ ∴ {tan}^{- 1} ( {y}_{2}^{′} ) - {tan}^{- 1} ( \frac{x}{y} ) = λ = {tan}^{- 1} ( \frac{{y}^{′} - \frac{x}{- y}}{1 + {y}^{′} \cdot \frac{x}{- y}} ) \\ ∴ {y}^{′} + \frac{x}{y} = k ( 1 - {y}^{′} \cdot \frac{x}{y} ) \\ [ where k = tan λ ] \\ {y}^{′} ( y + \frac{k x}{y /} ) = y k - \frac{x}{y /} \\ \frac{d y}{d x} = \frac{k y - x}{k y + x} \\ let y = v x \\ ∴ \frac{d y}{d x} = \frac{x d v}{d x} + v = \frac{kv x / - x /}{k x / + v x /} \\ \frac{x d v}{d x} + v = \frac{k v - 1}{k + v} - \frac{v ( k + v )}{( k + v )} = \frac{k / v - 1 - k / v - {v}^{2}}{( k + v )} \\ ∴ ( \frac{v + k}{{v}^{2} + 1} ) d v = \frac{- d x}{x} = ( \frac{2 v}{{v}^{2} + 1} ) \frac{d v}{2} + \frac{k d v}{{v}^{2} + 1} \\ Solution \text{:} k {tan}^{- 1} ( \frac{y}{x} ) + \frac{1}{2} ln ( \frac{{y}^{2}}{{x}^{2}} + 1 ) = {C}_{1} - ln ( x ) \qquad [ Math . 2 ]$$

On one hand, we want to minimize the default angle λ, so as to minimize the friction forces acting on the mechanism. On the other hand, we also want the tracks 071 and 072 to cover the distance between the two tracks 059 and 070 in the minimal possible rotation of the arm 004 or 006, for which the λ must be as large as possible. The relative distance between the slots 059 and 070, as well as the width of the tracks themselves along with the diameter of protrusion 038 should be as large as possible in order to make the device robust. The larger this distance between the slots also means a more robust inter-locking mechanism between arms 004, 005, 006 and 007 for various modes of operation, including scissor and grasper. However, since the over-all diameter of the tool is limited, which may be 8 mm for robotic laparoscopic surgery, the absolute size of the side left wall and side right wall is also restricted. Thus, while keeping the outer diameter limited to a certain value, but at the same time, increasing the track width, or increasing the distance between the two tracks, results in a higher ratio of the radius of outer track to the radius of inner track. Geometrically, this higher ratio of radius of outer track to inner track gives rise to a higher angle λ of the curve, given that the protrusion 038 of arms 004 or 006 must achieve this shift in their track within a limited angle of rotation about axis 017. A higher requirement of minimum angle λ implies a higher amount of friction that the arm 004 or 006 must overcome in order to make the shift between the scissor and grasper modes. The most efficient compromise between these factors will depend upon the strength of the material used, the coefficient of friction between the material of parts used, the ability of any nontoxic water based lubricants to effectively reduce the amount of frictional forces arising on the various parts, and other such things.

Thus, as many combinations are possible, a few examples are displayed below. In all these combinations, k is the tangent of λ; i.e. k=tan(λ). The equation from [154] is solved to yield a table of data points indicating various coordinates of points that lie on the curve. A smooth curve is joined between these points to yield the most efficient path for tracks 071 and 072.

In the table below, k=0.5, showing Upper Curve 071

TABLE 1

| Angle (deg) | x-coordinate | y-coordinate | R (radius) |
|---|---|---|---|
| 0.000 | 0.000 | 1.000 | 1.000 |
| 5.440 | 0.100 | 1.050 | 1.055 |
| 10.410 | 0.200 | 1.089 | 1.107 |
| 15.026 | 0.300 | 1.118 | 1.157 |
| 19.366 | 0.400 | 1.138 | 1.206 |
| 23.487 | 0.500 | 1.151 | 1.255 |
| 27.430 | 0.600 | 1.156 | 1.302 |
| 31.229 | 0.700 | 1.155 | 1.350 |
| 34.910 | 0.800 | 1.146 | 1.398 |
| 38.494 | 0.900 | 1.132 | 1.446 |
| 42.001 | 1.000 | 1.111 | 1.494 |
| 45.447 | 1.100 | 1.083 | 1.544 |
| 48.848 | 1.200 | 1.049 | 1.594 |
| 52.217 | 1.300 | 1.008 | 1.645 |
| 55.568 | 1.400 | 0.960 | 1.697 |
| 58.915 | 1.500 | 0.904 | 1.752 |
| 62.273 | 1.600 | 0.841 | 1.808 |
| 65.658 | 1.700 | 0.769 | 1.866 |
| 69.086 | 1.800 | 0.688 | 1.927 |
| 72.580 | 1.900 | 0.596 | 1.991 |
| 76.165 | 2.000 | 0.493 | 2.060 |

In the table below, k=0.48 showing Upper Curve 071

TABLE 2

| Angle (deg) | x-coordinate | y-coordinate | R (radius) |
|---|---|---|---|
| 0.000 | 0.000 | 1.000 | 1.000 |
| 5.451 | 0.100 | 1.048 | 1.053 |
| 10.446 | 0.200 | 1.085 | 1.103 |
| 15.099 | 0.300 | 1.112 | 1.152 |
| 19.484 | 0.400 | 1.131 | 1.199 |
| 23.658 | 0.500 | 1.141 | 1.246 |
| 27.660 | 0.600 | 1.145 | 1.292 |
| 31.523 | 0.700 | 1.141 | 1.339 |
| 35.274 | 0.800 | 1.131 | 1.385 |
| 38.935 | 0.900 | 1.114 | 1.432 |
| 42.525 | 1.000 | 1.090 | 1.479 |
| 46.061 | 1.100 | 1.060 | 1.528 |
| 49.558 | 1.200 | 1.023 | 1.577 |
| 53.033 | 1.300 | 0.978 | 1.627 |
| 56.501 | 1.400 | 0.927 | 1.679 |
| 59.976 | 1.500 | 0.867 | 1.732 |
| 63.476 | 1.600 | 0.799 | 1.788 |
| 67.020 | 1.700 | 0.721 | 1.847 |
| 70.631 | 1.800 | 0.633 | 1.908 |
| 74.334 | 1.900 | 0.533 | 1.973 |
| 78.167 | 2.000 | 0.419 | 2.043 |

In the table below, k=0.55 showing Lower Curve 072

TABLE 3

| Angle (deg) | x-coordinate | y-coordinate | R (radius) |
|---|---|---|---|
| 0.000 | 0.000 | −1.000 | 1.000 |
| −3.218 | 0.055 | −0.970 | 0.972 |
| −6.649 | 0.109 | −0.936 | 0.942 |
| −10.338 | 0.164 | −0.897 | 0.912 |
| −14.354 | 0.218 | −0.853 | 0.880 |
| −18.793 | 0.273 | −0.801 | 0.847 |
| −23.809 | 0.327 | −0.742 | 0.811 |
| −29.666 | 0.382 | −0.670 | 0.771 |
| −36.890 | 0.436 | −0.581 | 0.727 |
| −46.825 | 0.491 | −0.461 | 0.673 |
| −65.585 | 0.545 | −0.248 | 0.599 |
| −32.080 | 0.600 | −0.957 | 1.130 |

In the table below, k=0.50 showing Lower Curve 072

TABLE 4

| Angle (deg) | x-coordinate | y-coordinate | R (radius) |
|---|---|---|---|
| 0.000 | 0.000 | −1.000 | 1.000 |
| −3.209 | 0.055 | −0.973 | 0.974 |
| −6.609 | 0.109 | −0.942 | 0.948 |
| −10.240 | 0.164 | −0.906 | 0.921 |
| −14.156 | 0.218 | −0.865 | 0.892 |
| −18.437 | 0.273 | −0.818 | 0.862 |
| −23.201 | 0.327 | −0.764 | 0.831 |
| −28.642 | 0.382 | −0.699 | 0.797 |
| −35.113 | 0.436 | −0.621 | 0.759 |
| −43.386 | 0.491 | −0.519 | 0.715 |
| −55.857 | 0.545 | −0.370 | 0.659 |
| 86.179 | 0.600 | 0.040 | 0.601 |

As we can see, the overall objective achieved in this invention is the capability of a more number of motions, than the number of inputs that are available. In real-number mathematical or logical functions, the number of input degrees of freedom always equals the number of output degrees of freedom. Hence, theoretically, the number of degrees of freedom available at the end-effector must always equal the number of independent inputs available. In practice, however, this limitation can be circumvented to produce useful results, and one way of achieving this is explained in the present invention. The generalizations that we can draw are explained below.

Physical systems generally function only within a limited domain of operation of any given degree of freedom. Thus, the space outside the bounds of these limited domains of operation remains unused, and hence may be available for different purposes. In engineering systems, this fact can be utilized, with the help of various mechanisms, to carry out other useful tasks, such as an extra motion, extra function, etc.

Correspondingly, in the example of the physical system explained in this document, i.e. the multi-functional articulated wrist-like surgical instrument, the various degrees of freedom at the input are represented by the rotation of the four pulley-connectors 082, 084, 081 and 083 about their own axes. The four corresponding degrees of freedom at the output are represented respectively by (i) the rotation of the entire end effector about shaft 022, (ii) main bracket rotation 024.1, (iii) rotation of arm 005 about axis 017 and lastly, (iv) rotation of arm 007 also about axis 017. Correspondingly, the limited domains of operation of various degrees of freedom in this example happen to be the limits of the angle within which each of these elements are restricted to rotate. Purely mathematically speaking, each of these elements can be infinitely rotated about their corresponding axes in either direction. However, only a finite range of rotation is sufficient for practical use, and hence, it is engineered to operate within this particular useful domain of operation. Taking example of the rotation of arms 005 and 007 about axis 017, and the rotation of main bracket about axis 018, a range of rotation, i.e. a domain of operation, of say 180° angle in each case, may be sufficient to carry out the required surgical functions and perform wrist-like motions and hence are engineered such. Similar practical limits exist also for rotation about shaft 022.

Rotation of any of these elements beyond the present example of 180° angle is of little or no practical utility for the original function of the instrument, and hence this space can be considered to be outside of the bounds of the limited domain of operation.

In the present embodiment, as an example, the unused space outside the typically useful domain of operation of arms 005 and 007 is utilized. In other words, their rotation about axis 017, beyond an angle of 180° is utilized to carry out an extra function of switching between the modes of scissor and grasper. In other embodiments, the spaces beyond the domains of operation of another one or a combination of any other degrees of freedom may be crafted to perform this switching function, or any other additional required functions, motions, etc.

### CONCLUSION, RAMIFICATIONS AND SCOPE

We thus see that many combinations of the above factors give rise to different best-cases of devices. However, the general principle of the utilization of the space beyond the domains of operation of any degree(s) of freedom remains the same core idea behind any such iteration. Additionally, the principle of utilizing characteristic slots to allow one such combination of degrees of freedom to be used beyond their typical domain of operation in order to achieve the switching between different functions such as scissor and grasper, also remains the same core idea behind more such iterations.

For example, in another embodiment of the invention, the switching between different modes may occur when the device is articulated beyond the typical domain of operation of the roll degree of freedom. In yet another embodiment of the invention, the switching between different modes may occur when the device is articulated beyond the typical domain of operation of the yaw degree of freedom. Similarly for pitch degree of freedom.

The specific mechanism used to achieve the scissor and grasper modes of operation may differ in different embodiments. A multi-functional instrument may be created with different functions not necessarily including both of scissor and grasper. However, each such combination will contain an element to realize the switching actions between the different functional modes. Such an element can be actuated by moving the instrument beyond its typical domain of operation of any of its degrees of freedom, in order to realize the switching actions.

Another embodiment may contain the cam surfaces 095 on the arms 004 or 006, and instead have a corresponding protrusion 038 on the side right wall 008 and side left wall 009.

The methods disclosed herein can be employed in any suitable application. For example, the methods disclosed herein can be employed in surgical instruments, manual or powered, hand-held or robotic, directly controlled or tele-operated, for open or minimally invasive (single or multi-port) procedures. Examples of such instruments include those with distal components that receive actuating inputs (e.g., for grip control functions, component orientation control functions, component position functions, etc.). Illustrative non-limiting examples include teleoperated or hand-held instruments that include stapling, cutting, tissue fusing, imaging device orientation and position control, high force grasping, biopsy, and end effector and orientation control.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “force” is to be construed as encompassing both force and torque (especially in the context of the following claims), unless otherwise indicated herein or clearly contradicted by context. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

As will be realized, the present invention is capable of various other embodiments and that its several components and related details are capable of various iterations, all without departing from the basic concept of the present invention. This may include various materials, alternate mechanisms, etc. Accordingly, descriptions will be regarded as illustrative in nature and not as restrictive in any form whatsoever. Modifications and variations of the system and apparatus described herein will be obvious to those skilled in the art. Such modifications and variations are intended to come within the scope of the appended claims

1. A surgical assembly comprising,
an end effector with a configurable set of arms, said set of arms capable of performing a first function in a first mode of operation, and capable of performing a second function in a second mode of operation,
a set of input links capable of moving said set of arms through a first portion of range of motion and capable of moving said set of arms through a second portion of range of motion, such that the second portion of range of motion realizes a first extra action on part of said set of arms.

2. The surgical assembly of claim 1, wherein:
said set of arms are capable of switching the configuration from said first mode of operation performing said first function to said second mode of operation performing said second function,
such that the second range of motion realizing said first extra action is that of switching from said first mode of operation to said second mode of operation.

3. The surgical assembly of claim 2, wherein:
said set of arms are capable of switching the configuration back from said second mode of operation performing said second function to said first mode of operation performing said first function,
and said input link is capable of moving said set of arms through a third portion of range of motion, such that the third portion realizes a second extra action on part of said arm members.

4. The surgical assembly of claim 3, wherein:
said second extra action is that of switching the configuration back from said second mode of operation to said first mode of operation.

5. The surgical assembly of claim 1 wherein:
said first portion of range of motion is a useful domain of operation for carrying out said functions, said second range of motion being outside the useful domain of operation.

6. The surgical assembly of claim 5 wherein:
said first extra action is the switching between said first mode of operation and said second mode of operation, such that the switching action is carried out by same set of said input links, whereby said instrument becomes compatible with legacy systems.

7. The surgical assembly of claim 1 wherein:
said end effector has at least one degree of freedom, said set of input links contains members that are equal in number to the number of degrees of freedom of the end effector, such that said first extra action is carried out without additional input link members.

8. The surgical assembly of claim 7, wherein:
said end effector has at least a second degree of freedom, and the movement of said set of arms through said first portion of range of motion and said second portion of range of motion, both occur over said second degree of freedom of said set of arms, such that the first extra action occurs during the movement of said set of arms over the second degree of freedom, also effecting a change in configuration of said set of arms.

9. The surgical assembly of claim 1 wherein:
said first function is that of scissor and said second function is that of grasper.

10. The surgical assembly of claim 9, wherein:
the set of arm members further includes a set of insulation layers, and an electrical connection, such that the instrument can function as a bipolar cautery while in the grasper mode.

11. The surgical assembly of claim 1 wherein:
said set of arms are rotatably connected to a hinge, and said movement of set of arms by said set of input links is that of rotation about said hinge.

12. The surgical assembly of claim 11 wherein:
said end effector includes a cam surface drivingly coupled with said set of arms and shaped to realize the first extra action when said set of input links rotate said set of arms through said second portion of the range of motion.

13. The surgical assembly of claim 12 wherein:
said cam surface includes a first track segment which has a centerline with a constant first radius relative to the hinge and a second track segment which has a centerline with a gradually changing radius relative to the hinge.

14. The surgical assembly of claim 13 wherein:
said end effector includes a follower, said cam surface further includes a third track segment concentric to the first track segment having a centerline with a constant second radius relative to the hinge, said second radius being different from said first radius, a fourth track segment with gradually changing radius, such that the second and fourth track segments smoothly join the first and third track segments, whereby said follower is able to shift from the first track segment to the third and back, by following the second and fourth track segments of the cam surface.

15. The surgical assembly of claim 14 wherein:
movement of said follower in the first and third track segment of the cam surface corresponds to said first and second modes of operation of the instrument and both also corresponding to the first portion of the range of motion by which the set of input links rotate said set of arms, and the movement of said follower in the second track segment of the cam surface corresponds to said second portion of the range of motion by which the set of input links rotate said set of arms.

16. The surgical assembly of claim 15, wherein:
movement of said follower in said second track segment of said cam surface realizes said first extra action, said first extra action being a change in the internal configuration of said set of arms whereby the arms switch the configuration between said first mode of operation of first function and said second mode of operation of second function, whereby said first extra action results in the instrument switching between performing said first function and said second function.

17. The surgical assembly of claim 16, wherein:
the shape of said second track segment of said cam surface follows an efficient profile of a characteristic curve, such that the movement of said follower occurs with minimum frictional opposing force as well as minimum rotation about said hinge, within the second portion of the range of motion.

18. The surgical assembly of claim 1, wherein:
said first function is that of a needle driver and said second function is that of a scissor.

19. The surgical instrument of claim 1, wherein:
said end effector has three degrees of freedom of roll, pitch and yaw, in addition to the actuation of said end effector.

20. The surgical instrument of claim 19, wherein:
the movement of said set of arms through said first portion of range of motion and said second portion of range of motion, both occur over said degree of freedom of roll, of said set of arms, such that the first extra action occurs during the movement of said set of arms over said degree of freedom of roll.

21. The surgical instrument of claim 19, wherein:
the movement of said set of arms through said first portion of range of motion and said second portion of range of motion, both occur over said degree of freedom of pitch, of said set of arms, such that the first extra action occurs during the movement of said set of arms over said degree of freedom of pitch.

22. The surgical assembly of claim 19, wherein:
the movement of said set of arms through said first portion of range of motion and said second portion of range of motion, both occur over said degree of freedom of yaw, of said set of arms, such that the first extra action occurs during the movement of said set of arms over said degree of freedom of yaw.

23. The surgical assembly of claim 1, wherein:
said set of input links is a set of mechanical coupler links in the form of a wire-rope and pulley system.

24. The surgical assembly of claim 1, wherein:
said first extra action realizes a motion within said set of arms in order to effect a change in the configuration.

25. The surgical assembly of claim 24, wherein:
said change of configuration of the set of arms results in a change of the functional mode of operation of the instrument.

26. A method of treating tissue comprising:
moving a configurable set of arms of an end effector of a surgical instrument through a first portion of a range of motion and a second portion of range of motion, configuring said set of arms so as to enable them to function as a scissor and cut the tissue, and configuring said set of arms so as to enable them to function as a grasper and grasp the tissue respectively, such that said second portion of a range of motion realizes an extra action on part of the arm members.

27. The method of claim 26, wherein:
said extra action is that of switching between the modes of scissor and grasper.
